(19) United States
(12) Plant Patent Application Publication
(10) Pub. No.: US 2004/0163153 P1
(43) Pub. Date: Aug. 19, 2004

(54) Aglaonema plant named 'Moonlight Bay'

(76) Inventor: Richard J. Henny, Apopka, FL (US)

Correspondence Address: Thomas D. Stadsklev, Florida Foundation Seed Producers, Inc., P.O. Box 309, Greenwood, FL, 32443, US

(21) Appl. No.: 10/365,786

(22) Filed: Feb. 14, 2003

(51) Int. Cl. A01H 5/00

(52) U.S. Cl. PLT/376

(57) ABSTRACT

A new and distinct cultivar of Aglaonema named ‘Moonlight Bay’, characterized by its unique leaves that display three distinct colors consisting of a dark green background and two distinct lighter gray-green areas that appear in irregular splashes. The lightest gray-green areas of the leaf surface tend to center around the midrib in irregular splashes while the darker gray-green areas follow the lateral leaf veins from the midrib to the leaf margin. The stems and petioles each display two distinct colors. Plants are mostly symmetrical, relatively upright and outwardly arching in plant habit and suitable for 15 to 30 cm containers.

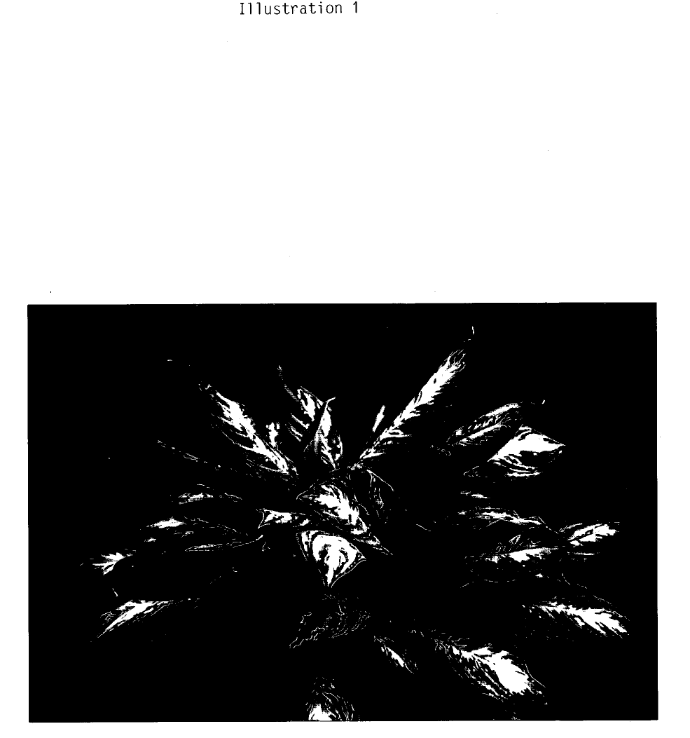

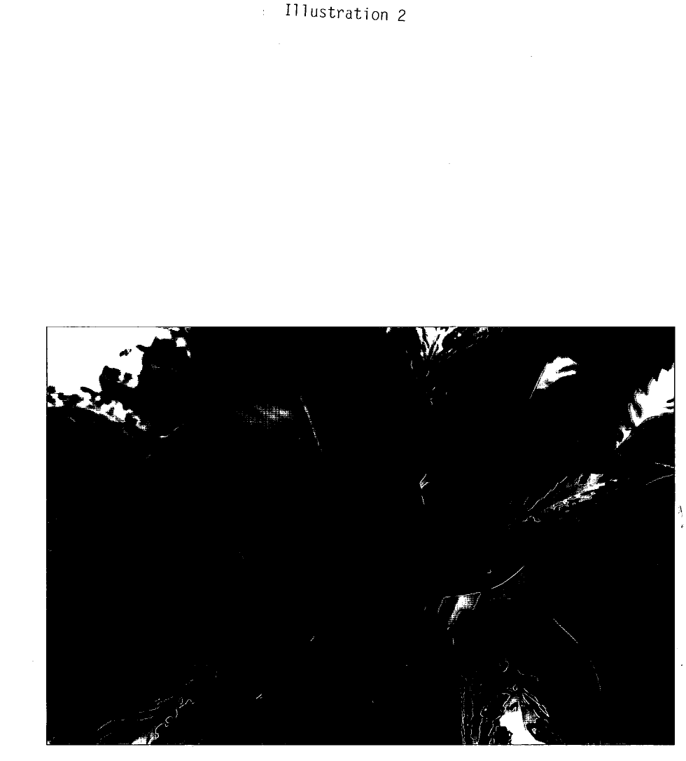

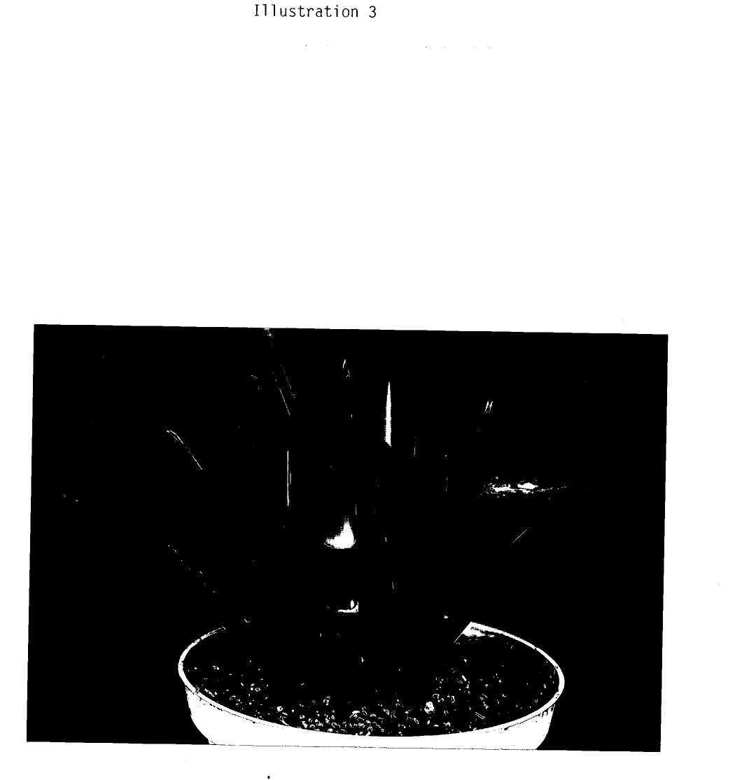

1. The upper surface of mature leaves display three distinct colors consisting of a dark green background and two distinct lighter gray-green areas that appear in irregular but consistent patterns.

2. The lightest gray-green areas of the leaf surface tend to center around the midrib in irregular splashes while the darker gray-green areas follow the lateral leaf veins from the midrib to the leaf margin.

3. The stems and petioles each display two distinct colors.

4. Plants are relatively upright and outwardly arching in plant habit.

5. Plants are mostly symmetrical, relatively compact and suitable for 15 to 30 cm containers.

6. Plants are moderate in branching, averaging 2-3 basal shoots when grown from stem cuttings in controlled tests.

### DESCRIPTION

The present invention relates to a new and distinct cultivar of Aglaonema plant, botanically known as Aglaonema hybrid, and hereinafter referred by the cultivar name ‘Moonlight Bay’.

The new Aglaonema is a product of an extensive University of Florida ornamental tropical foliage plant breeding program conducted by the inventor, Dr. Richard J. Henny in Apopka, Fla. The objective of the breeding program is to develop new Aglaonemas with improved growth habits, performance and novel appearance.

‘Moonlight Bay’ was discovered and selected by the inventor as a sport from a large population of tissue-culture propagated Aglaonema ‘Silver Bay’ (a previously developed University of Florida hybrid cultivar) growing in a greenhouse in Apopka, Fla. Compared to plants of the original parent cultivar ‘Silver Bay’, leaves of mature plants of the new Aglaonema display a unique foliar variegation patter consisting of three distinct colors on the leaf surface. The large central gray pattern of ‘Silver Bay’ has been replaced by a much smaller and more irregular pattern of the same color. The darker gray background pattern is similar to ‘Silver Bay’ but is much more exposed due to the smaller area of lighter gray that no longer masks it in the new cultivar. ‘Moonlight Bay’ petioles and stems each display two distinct colors and plants are somewhat less compact than ‘Silver Bay’.

Asexual propagation of the new cultivar by division, stem cuttings and tissue culture at Apopka, Fla. since 1999 has shown that the unique features of this new Aglaonema plant are stable and reproduced true to type in successive generations.

The following traits have been repeatedly observed and are determined to be the unique characteristics of ‘Moonlight Bay’. These characteristics in combination distinguish ‘Moonlight Bay’ as a new and distinct cultivar from other similar Aglaonema cultivars.

1. The upper surface of mature leaves display three distinct colors consisting of a dark-green background and two distinct lighter gray-green areas that appear in irregular but consistent patterns.

2. The lightest gray-green areas of the leaf surface tend to center around the midrib in irregular splashes while the darker gray-green areas follow the lateral leaf veins from the midrib to the leaf margin.

3. Plants of ‘Moonlight Bay’ are relatively upright and somewhat outwardly arching in plant habit.

4. Plants of ‘Moonlight Bay’ are fairly symmetrical and relatively compact and are suitable for 15 to 30-cm containers.

5. Plants of ‘Moonlight Bay’ are moderate in branching, averaging 2-4 basal shoots when grown from stem cuttings in controlled tests.

6. Plants of ‘Moonlight Bay’ have stems and petioles that each display two distinct colors.

### BRIEF DESCRIPTION OF THE DRAWINGS

The three color photographic drawings were taken from a typical plant of Aglaonema ‘Moonlight Bay’ grown in a 20 cm diameter pot (3.9 liter volume), approximately 1 year after planting a 12-week-old rooted stem cutting with 5 leaves and grown under appropriate growing conditions. Colors are as accurate as possible with color illustrations of this type.

1. The first drawing depicts a top perspective view of a plant of ‘Moonlight Bay’ showing the unique foliar variegation pattern.

2. The second drawing depicts a closeup of the top and underside of mature leaves to show how the upper leaf surface variegation is not visible on the lower leaf surface.

3. The third drawing shows the petiole and stem color of a typical stem from a mature plant.

The following observations, measurements and comparisons describe plants grown in Apopka, Fla., in a shaded greenhouse and under conditions which closely approximate those used in horticultural practice. Plants were grown under day temperatures ranging from 21 to 38 degrees C. and night temperatures ranging from 7 to 21 degrees C. The greenhouse shade provided approximately a 70 to 80 percent decrease in ambient light level. In the following description, color references are made to The Royal Horticultural Society Colour Chart.

Origin: Sport of Aglaonema ‘Silver Bay’ (not patented).

Classification: Aglaonema cultivar ‘Moonlight Bay’.

Propagation: Asexual propagation either by stem cuttings, tissue culture or division.

Plant:

In a 20 cm pot for a plant grown from a 12 week-old-cutting under appropriate growing conditions for 9 months, ‘Moonlight Bay’ has an average canopy width of 54.0 cm and a canopy height of 34.0 cm. Mature leaves average 30.2 cm in length and 11.6 cm in width. Plants average 2-3 basal shoots.

Stem:

Growth pattern.—The stem is erect in growth and is 2.0 to 3.0 cm in diameter at five (5) cm above the soil surface. Internode distance is approximately 1.5 to 2.0 cm at five (5) cm above the soil.

Color.—The stem consists of two different colored areas that run together. The darker areas that are exposed to light are yellow green (RHS 146B) and roughly triangular in shape. These blend with areas of the stem that are a lighter shade of yellow green (RHS 152B/C). The lighter areas are normally hidden by the attached clasping petiole.

Petiole: The following information is based on the 5^{th} expanded leaf from the apex of a one-year-old plant.

Dimensions.—Overall petiole length ranges from 12.0 to 20.0 cm. The petiole has expanded fleshy edges extending from the midrib and referred to as wings. The wings are approximately 2.0 to 2.4 cm wide, one-half the distance from the petiole base to the wing apex. The petiole wings average 10.0 to 16.0 cm in length and extend from the petiole base to within 0.5 to 5.0 cm of the leaf blade. The wings roll inward and the edges come to within 1.0 to 5.0 mm of touching when not in contact with the stem. The angle between the stem and petiole is approximately 30 degrees from the stem axis for newer leaves. As leaves mature the angle becomes 40 to 50 degrees. The petiole and the leaf midrib form a near straight line on newly unfolding leaves. With maturity the leaf blades arch down to form an angle of 45 to 60 degrees between the petiole and the leaf underside. In some cases the oldest lower leaves may arch far enough to form an angle of 90 degrees.

Color.—The petiole wings consist of two colors including yellow-green (RHS 146A/B) on the outside edges blending to a lighter yellow-green (RHS-148C) in the center. The center petiole color extends up the leaf midrib on the leaf underside.

Leaf:

Growth pattern.—‘Moonlight Bay’ leaves are elliptic and asymmetric in shape. Leaf bases are obtuse, tips are acuminate, and the margins are entire. There is some undulation in the leaves.

Dimensions.—Mature leaves may reach a length of 34 cm and a width of 13 cm although leaves for the pot size and indicated average 29.0 to 31.0 cm in length and 11.0 to 13.0 cm in width (length/width ratio of 2.4 to 2.6).

Midrib.—The midrib is prominent and slightly recessed on the upper leaf surface.

Color.—The upper surface of mature leaves display three distinct colors consisting of a darker green background that is closest to (RHS 147A) and two distinct lighter gray-green areas that appear in irregular but consistent patterns. The lightest gray-green (RHS 191A) areas of the leaf surface tend to center around the midrib in irregular splashes which vary is size from 0.1 to 0.2 cm to 5.0 to 7.0 cm at the longest point. Most of the lighter colored splashes are concentrated on either side of the midrib and do not extend out beyond half the distance to the leaf margin. The largest splashes of light gray-green normally are near the leaf tip. Larger darker gray-green (RHS 189A) areas follow the lateral leaf veins from the midrib to the leaf margin. These areas can be up to 2.0 cm wide, are irregular in shape have defined edges, and contain minute specks (less than 0.5 mm) of dark green (RHS 147A). The leaf midrib is the same color (RHS 191A) where the lighter splashes overlap it. In other areas the midrib is finely striped consisting of lighter gray-green (RHS 191A) and the darker gray-green (RHS 189A). The leaf underside is a uniform medium yellow-green (RHS 147B).

Inflorescence: Although the Aglaonema inflorescence has no commercial value the following description applies. Spathes and spadices are held on arching peduncles and there may be 3 to 6 inflorescences produced at each flowering node. Spathes range in length from 7.0 to 9.0 cm in length and 1.0 to 1.5 cm in width. With the spathe removed, the female portion of the inflorescence ranges from 1.0 to 2.0 cm in length and the male portion may be 4.0 to 5.0 cm in length. The number of female flowers present per inflorescence ranges from 14 to 23. The outer spathe color is yellow-green (RHS 144A). Again, with the spathe removed, the male portion of the spadix is grayed-orange (RHS 163C) in new inflorescences and darkens to grayed-orange (RHS 165B) with age. The female flower stigma is yellow (RHS 13B) while the female ovary is yellow-white (RHS 158B).

Seed: No seed has been observed on ‘Moonlight Bay’.

Roots: Roots are typical for Aglaonema being thick white roots with fine laterals.

Disease/Insect resistance: In trial tests under commercial production conditions plants of Aglaonema ‘Moonlight Bay’ have not shown any unusual susceptibility to diseases or insects common to Aglaonema.

I claim:

1. A new and distinct cultivar of Aglaonema named ‘Moonlight Bay’, characterized by its unique leaves that display three distinct colors consisting of a dark green background and two distinct lighter gray-green areas that appear in irregular splashes. The lightest gray-green areas of the leaf surface tend to center around the midrib in irregular splashes while the darker gray-green areas follow the lateral leaf veins from the midrib to the leaf margin. The stems and petioles each display two distinct colors. Plants are mostly symmetrical, relatively upright and outwardly arching in plant habit and suitable for 15 to 30 cm containers.
